(19) United States
(12) Patent Application Publication
(10) Pub. No.: US 2016/0324201 A1
(43) Pub. Date: Nov. 10, 2016

(54) SWEETENER COMPOSITION FOR ALLEVIATING DIABETES, CONTAINING SLOWLY DIGESTIBLE INGREDIENT

(71) Applicant: CJ CHEILJEDANG CORPORATION, Seoul (KR)

(72) Inventors: Young Jae Kim, Seoul (KR); Jin Hee Park, Goyang-si (KR); Min Hae Kim, Incheon (KR); Seong Bo Kim, Seoul (KR); Se Hee Hwang, Seoul (KR); Young Mi Lee, Bucheon-si (KR)

(21) Appl. No.: 15/215,871

(22) Filed: Jul. 21, 2016

Related U.S. Application Data
(63) Continuation of application No. 14/345,155, filed on Mar. 14, 2014.

(30) Foreign Application Priority Data
Sep. 15, 2011 (KR) 10-2011-0092801

(51) Int. Cl. A23L 27/30; A61K 31/715; A23L 2/60; A61K 31/7004; A23L 33/125; A23L 33/00

(52) U.S. Cl. CPC A23L 27/34; A23L 33/125; A23L 33/30; A23L 2/60; A61K 31/7004; A61K 31/715; A23V 2002/00

(57) ABSTRACT

The present invention relates to a sweetener composition for alleviating diabetes, containing psicose and a slowly digestible or digestion-resistant polysaccharide as active ingredients, wherein diabetes alleviating effects and the quality of sweetness are enhanced.

### CROSS REFERENCE TO RELATED APPLICATIONS

The present application is a continuation application of U.S. application Ser. No. 14/345,155 filed Mar. 14, 2014, which is a U.S. national phase application of PCT/KR2012/007425 filed Sep. 17, 2012, which claims priority to KR Application No. 10-2011-0092801, filed Sep. 15, 2011. U.S. application Ser. No. 14/345,155 is herein incorporated by reference in its entity.

### TECHNICAL FIELD

The present invention relates to a sweetener composition for alleviating diabetes containing D-psicose and a slowly digestible polysaccharide or digestion-resistant polysaccharide as active ingredients.

### BACKGROUND ART

Sugar contains sucrose as a main ingredient and is one of representative sweeteners exhibiting sweet taste upon adding to food. Sugar has outstanding sweetness and thus has been considered as one of the most preferred sweeteners which are added to various foods and processed foods to improve the food taste and stimulate appetite. However, recently, as the harmful effects of sugar have been revealed, problems concerning use thereof are being reported. Specifically, excessive sugar intake is a major cause of tooth decay as well as various lifestyle related diseases such as obesity, diabetes, and the like. For these reasons, there is global demand for an alternative sugar as a replacement for sugar.

Diabetes is a class of metabolic diseases due to lack of secreted insulin or because secreted insulin does not work effectively. Diabetes is characterized by high blood glucose levels which causes various symptoms and leads to discharge of glucose in the urine. There are two types of diabetes—Type 1 and Type 2. Type 1 diabetes is called “juvenile diabetes” and results from the body's failure to produce insulin. In Type 2 diabetes, not enough insulin is produced. Type 2 diabetes results from insulin resistance, a condition in which cells fail to burn glucose effectively due to weakened insulin response, raising blood glucose levels. It is known that Type 2 diabetes is greatly affected by environmental factors, such as high caloric, high fat and high protein diet, lack of exercise, stress and the like. In addition, it is known that diabetes can also be caused by defects in specific genes or pancreatic surgery, infection, drugs, and the like.

In diabetics, although hyperglycemic symptoms are generally the most pronounced and noticeable, hypoglycemic symptoms can be just as fatal as hyperglycemic symptoms. In a person having normal blood glucose levels, the person generally exhibits hypoglycemic symptoms when fasting blood glucose level drop to 55 mg/dL or less. However, it is known that diabetic patients may exhibit hypoglycemic symptoms even if patients have blood glucose levels higher than 70 mg/dL. Accordingly, unconditional provision of a sweetener having low calories or provision of a sweetener having an ability to inhibit the absorption of sugars may lead to unexpected negative reactions owing to excessively decreased blood glucose level in diabetic patients.

In this context, there is a strong need for an improved sweetener which has a suitable sweetness to replace sugars and low calories, thereby preventing excessive sugar intake by simply inhibiting the absorption of sugars.

D-psicose is an epimer of D-fructose and is a sub-category of functional sugars known as rare sugars. It is known that D-psicose has a high degree of sweetness equivalent to about 60% to 70% that of sugar and has close to zero calories, and thus is effective in preventing or treating adult diseases such as obesity and the like. In addition, D-psicose is also known to have efficacy to prevent and treat diabetes since D-psicose is able to inhibit the absorption of sugars such as glucose, D-fructose and the like. Furthermore, D-psicose is known to have excellent solubility and thus takes keen attention for application to foods.

D-psicose has a relatively good sweetness, but has a relatively low sweetness as compared with sugar. In this regard, the use of D-psicose alone as a sweetener for food additives cannot satisfy consumers accustomed to the taste of sugar, thereby hindering market acceptance. In order to overcome such problems stemming from the use of D-psicose alone, namely, in order to use D-psicose alone while attaining sweetness satisfying general consumers, it is inevitable to increase the amount of D-psicose, which can provide excessive thick feeling to foods utilizing D-psicose, thereby causing deterioration in texture of foods.

On the other hand, digestion-resistant maltodextrin is a class of dietary fiber (polysaccharides). As can be seen from its name, the digestion-resistant maltodextrin is a dietary fiber which is indigestible in the human body, and is characterized by having a high molecular weight carbohydrate structure having a degree of polymerization higher than general maltodextrins.

As the prior art related to the present invention, there are Korea Patent Publication No. 10-2011-0035805A (published on Apr. 6, 2011), Korea Patent No. 10-0815212 B1 (published on Mar. 19, 2008), Korean Patent No. 10-0910081 B 1 (published on Jul. 30, 2009), and the like.

### DISCLOSURE

### Technical Problem

The present invention is aimed at providing a sweetener composition for alleviating diabetes containing D-psicose and a slowly digestible polysaccharide or a digestion-resistant polysaccharide as active ingredients.

The inventors of the present invention have found that, when D-psicose which is a low calorie sweetener and known to have efficacy in preventing and alleviating diabetes is used alone, D-psicose cannot effectively perform a role as a sweetener due to declined sweetness. For this reason, various attempts have been made to produce a sweetener having improved sweetness by mixing D-psicose with other sugars or sugar alcohols having excellent sweetness such as tagatose, xylose, xylitol, and the like. However, it was found that such mixing can excessively inhibit digestion and absorption of sugars introduced into the body, and thus can cause an excessively low level of blood glucose, which in turn leads to side effects fatal to diabetic patients.

The inventors have developed a sweetener composition for alleviating diabetes using D-psicose and a slowly digestible polysaccharide or a digestion-resistant polysaccharide in combination, which may provide advantages including slow digestion of glucose in the body, allowing blood glucose levels to elevate gradually after meals, thereby preventing hyperglycemic symptoms in which the blood glucose level in diabetic patients is sharply increased, while preventing hypoglycemic symptoms fatal to diabetic patients by supplying sugars slowly but consistently and appropriately.

### Technical Solution

In accordance with one embodiment of the present invention, a sweetener composition for alleviating diabetes contains D-psicose and a slowly digestible polysaccharide or a digestion-resistant polysaccharide as active ingredients.

In accordance with another embodiment, the slowly digestible polysaccharide or the digestion-resistant polysaccharide may include at least one polysaccharide selected from the group consisting of palatinose, trehalose, digestion-resistant maltodextrins and oligosaccharides.

In accordance with a further embodiment of the present invention, the sweetener composition for alleviating diabetes may further include a high-intensity sweetener.

In accordance with yet another embodiment of the present invention, the high-intensity sweetener may include at least one selected from the group consisting of steviol glycoside, sucralose, aspartame, Siraitia grosvenori extract, Glycyrrhiza uralensis Fischer extract, thaumatin, and agave syrup.

In accordance with yet another embodiment of the present invention, the slowly digestible polysaccharide or the digestion-resistant polysaccharide may be present in an amount of 0.01 to 200 times the weight of D-psicose, and the high-intensity sweetener may be present in an amount of 0.001 to 2 times the weight of D-psicose.

### Advantageous Effects

Using D-psicose and a slowly digestible polysaccharide or a digestion-resistant polysaccharide in combination, the present invention provides a sweetener composition for alleviating diabetes, which may allow very slow digestion of glucose in the body to provide gradual increase in blood glucose level, thereby preventing hyperglycemic symptoms in which the blood glucose levels in diabetic patients are sharply increased, while preventing hypoglycemic symptoms fatal to diabetic patients by supplying sugars slowly but consistently and appropriately without excessively inhibiting the supply of sugars.

In addition, according to another embodiment, the present invention provides a sweetener composition, which includes D-psicose and a slowly digestible polysaccharide or a digestion-resistant polysaccharide in a specific ratio, and thus has outstanding properties in terms of alleviation of diabetes as compared with sweetener compositions prepared in other optional ingredient ratios.

According to a further embodiment, the present invention provides a sweetener composition for alleviating diabetes, which can reduce calories while increasing sweetness and has improved quality of sweetness by adding a specific high-intensity sweetener.

According to yet another embodiment, by employing sucralose among high-intensity sweeteners, the present invention provides a sweetener composition for alleviating diabetes, which exhibits sweetness similar to sugar while providing a low glycemic index and substantially zero calories to allow excellent short term or long term blood glucose control in Type 1 or Type 2 diabetic patients.

### BRIEF DESCRIPTION OF THE DRAWINGS

FIG. 1 is a graph depicting change of blood glucose in Experimental Example 1.

FIG. 2 is a graph depicting change of area under the curve of glucose in Experimental Example 2.

### MODE FOR INVENTION

Hereinafter, the present invention will be described in more detail. Descriptions of details apparent to those skilled in the art having ordinary knowledge in this technical field or relevant fields will be omitted herein.

The present invention provides a sweetener composition for alleviating diabetes, which contains D-psicose and a slowly digestible polysaccharide or a digestion-resistant polysaccharide as active ingredients.

The term “slowly digestible” means a property that digestion is performed at a slow speed in the small intestine of the human body.

The term “digestion-resistant” means a property that digestion is not easily performed in the small intestine of the human body.

Another embodiment of the present invention provides a sweetener composition for alleviating diabetes, including at least one polysaccharide selected from the group consisting of palatinose, trehalose, digestion-resistant maltodextrins and oligosaccharides as the slowly digestible polysaccharide or the digestion-resistant polysaccharide.

Polysaccharide refers to a saccharide formed by binding two or more monosaccharide units, namely, a saccharide higher than a disaccharide.

Palatinose is a natural disaccharide found in nature. Palatinose may be obtained from honey, sugar cane or the like. Industrially, palatinose may be produced by treating sugar with an enzyme capable of changing the structure of sugar. Palatinose has a disaccharide structure in which a glucose molecule and a fructose molecule are bound.

When consumed together with a saccharide such as sugar or starch or the like, palatinose may provide an effect of retarding absorption of saccharide. Palatinose is digested at a slow rate corresponding to ⅕ that of sugar. Namely, since digestion and absorption of palatinose are performed at a slow rate and increase in blood glucose level is avoided, palatinose provides effects of inhibiting secretion of insulin and accumulation of fat.

Trehalose is a class of storage carbohydrates and mainly found in various bacteria, fungi, yeasts, insects, animals, plants, and the like. Trehalose is a non-reducing disaccharide, in which two molecules of glucose are bound.

The digestion-resistant maltodextrin is a class of dietary fibers and refers to a polysaccharide that is indigestible in the body.

Oligosaccharides refer to polysaccharides in which two or more monomer units are bound through a glycosidic bond. Specifically, since fructooligosaccharides, galactooligosaccharides, soybean oligosaccharides, branched oligosaccharides, lactooligosaccharides, chitooligosaccharides, gentiooligosaccharides, and the like corresponding to digestion-resistant oligosaccharides are not digested by digestion enzymes in the body, there is no rise in blood glucose levels after intake of oligosaccharides. Accordingly, oligosaccharides do not substantially affect blood glucose levels and the absorption of organic acids generated by enteric fermentation does not substantially affect blood glucose levels.

Oligosaccharides used in the present invention are not limited in terms of the number of bonded monosaccharides (for example, disaccharides, trisaccharides, tetrasaccharides, pentasaccarides, and the like), and reducing or non-reducing oligosaccharides are utilizable.

A further embodiment of the invention provides a sweetener composition for alleviating diabetes further including a high-intensity sweetener.

The high-intensity sweetener refers to a sweetener exhibiting high sweetness several to hundreds times that of sugar.

The high-intensity sweetener is not particularly limited, but the high-intensity sweetener may include at least one selected from steviol glycoside, sucralose, aspartame, Siraitia grosvenori extract, Glycyrrhiza uralensis Fischer extract, thaumatin, and agave syrup.

Steviol glycoside refers to a material obtained by processing a water soluble extract of the leaf of Stevia rebaudiana. Preferably, rebaudioside A (Reb A) is used as the steviol glycoside. When rebaudioside A is used as a high-intensity sweetener, the bitter taste, metallic taste and the like characteristic of high-intensity sweeteners such as steviol glycoside may be avoided, thereby providing a sweetener composition for preventing and treating obesity, which has excellent taste.

Sucralose refers to a synthetic analog of sugar obtained by substituting hydroxyl groups of sugar with chlorines.

Aspartame refers to an amino acid type synthetic sweetener prepared from phenylalanine and aspartic acid.

Siraitia grosvenori extract refers to an extract obtained from fruits of Siraitia grosvenori, which is a perennial herb belonging to the family of Cucurbitaceae. The extract used in the present invention is not particularly limited as to extraction method. Any extract prepared by methods known in the technical field of the present invention or similar field may be used.

Glycyrrhiza uralensis Fischer extract refers to an extract obtained from Glycyrrhiza uralensis, which is a perennial herb belonging to genus Cassia. The extract used in the present invention is not particularly limited as to its extraction method. Any extract prepared by methods known in the technical field of the present invention or similar field may be used.

Thaumatin refers to a protein type sweetener obtained from fruits of Thaumatococcus daniellii, followed by purifying the extract. The extract is not particularly limited as to extraction method, and any extract prepared by methods known in the technical field of the present invention or similar field may be used.

Agave syrup refers to syrup from Blue Tequilana Webre Agave, which belongs to a class of Agave deserti Engelm. Agave syrup used in the present invention is not particularly limited, but a natural organic product in syrup form obtained by collecting six-year-old Blue Tequilana Webre Agave, extracting the collected Blue Tequilana Webre Agave to provide juice, and heating the juice to an appropriate temperature to concentrate the same.

Agave syrup is a natural sweetener having sweetness 1.5 times that of general sugar and a glycemic index (GI) of about ⅓ that of general sugar. The main ingredient of agave syrup is inulin, corresponding to a collection of polysaccharides, wherein the polysaccharide refers to a class of saccharides in which fructose polymers known as fructans, which are dietary fibers, are consecutively linked. Unlike fructose, inulin substantially does not affect blood glucose and insulin, and does not increase triglyceride levels. Therefore, agave syrup is suitable for use in diabetic patients and has effects that may aid to prevent and control diseases related to blood glucose levels. Additionally, agave syrup may contain minerals such as iron, calcium, potassium, magnesium, and the like.

According to yet another embodiment, by employing sucralose among high-intensity sweeteners, the present invention provides a sweetener composition for alleviating diabetes, which exhibits sweetness the most similar to sugar while providing a low glycemic index and substantially zero calories to allow excellent short term or long term blood glucose control in Type 1 or Type 2 diabetic patients.

According to yet another embodiment, the sweetener composition for alleviating diabetes contains the slowly digestible polysaccharide or the digestion-resistant polysaccharide in an amount of 0.01 times to 200 times, preferably 0.01 times to 100 times, more preferably 0.1 times to 50 times the weight of D-psicose.

Further, the sweetener composition for alleviating diabetes contains the high-intensity s sweetener in an amount of 0.001 times to 2 times, preferably 0.001 times to 1.5 times, more preferably 0.001 times to 1 times the weight of D-psicose.

Within this content range, the sweetener may have advantages in that problems of sharp rise in blood glucose level upon ingesting the sweetener or hypoglycemic symptoms in which fasting blood glucose level is excessively declined may be inhibited and the sweetener has sweetness similar to sugar.

Hereinafter, the present invention will be described in more detail with reference to Examples, Comparative Examples and Experimental Examples. These examples are provided for illustration only and are not to be in any way construed as limiting the present invention.

### EXAMPLES 1, 2 AND COMPARATIVE EXAMPLES 1 TO 3

### Preparation of Sweetener Composition

Sweetener compositions as listed in Table 1 were prepared.

In Comparative Example 1, 5 g of sugar was used. Sweetener compositions of Comparative Examples 2 and 3, and Examples 1 and 2 were prepared by formulating the components as listed in Table 1 such that the compositions exhibited sweetness similar to that of 5 g of sugar.

TABLE 1

| | | Relative | | | | | |
|---|---|---|---|---|---|---|---|
| | | Sweetness | | | | | |
| Raw material | | per g of raw | Com. | Com. | Com. | Ex. | Ex. |
| (g) | Manufacturer | material | Ex.1 | Ex. 2 | Ex. 3 | 1 | 2 |
| Sugar | CJ Cheiljedang | 1 | 5 | — | — | — | — |
| Erythritol | Zivogreen | 0.63 | — | 8 | — | — | — |
| D-psicose | CJ Cheiljedang | 0.56 | — | — | 9 | 8 | 4 |
| Digestion-resistant | Matsudani Korea | 0.1 | — | — | — | 5.6 | 3.5 |
| maltodextrin | Ltd. | | | | | | |
| Rebaudioside A | GLG | 200 | — | — | — | — | 0.015 |

| | | | | | |
|---|---|---|---|---|---|
| Relative sweetness of sweetener composition | 5 | 5.04 | 5.04 | 5.04 | 5.03 |

Among the sweetener compositions, erythritol used in Comparative Example 2 corresponds to a material that is substantially zero calories and provides only sweetness without affecting blood glucose level. Rebaudioside A used in Example 2 is a steviol glycoside and is a natural high-intensity sweetener prepared by extracting components having sweet taste from steviol glycosides.

### Experimental Example 1

### Measurement of Changes in Blood Glucose

(1) Preparation of Test Specimen for Measuring Blood Glucose and Method of Intake

In order to measure changes in blood glucose level after consuming each sweetener composition prepared in Comparative Examples 1 to 3 and Examples 1 and 2, the following experiment was carried out for a normal group having a fasting glycemic index of 100 mg/dL or less and consisting of five males and five females in their twenties to forties.

The sweetener composition intake was conducted by providing subjects with an identical meal and then allowing them to drink coffee containing the sweetener composition. The meals given to the subjects are as listed in Table 2.

TABLE 2

| Food materials | Amount used (g) |
|---|---|
| Bread | 75 |
| Ham | 20 |
| Lettuce | 20 |
| Strawberry jam | 20 |
| Crabstick | 30 |
| Cheddar cheese | 10 |

As shown in Table 2, the meal given to the subjects consisted of 75 g of bread, 20 g of lettuce, 20 g of strawberry jam, 30 g of crabstick and 10 g of cheddar cheese. As a result of analysis using Korean food composition table (CanPro 3.0, The Korean Nutrition Society), it was found that the meal had a total caloric content of 356.4 kcal, which consisted of 59.57% of sugar, 18.14% of protein and 22.27% of lipid.

After the meal, the subjects drank coffee made by mixing 1.6 g of sugar free coffee and the sweetener composition prepared in Comparative Examples 1 to 3 and Examples 1 and 2 in 200 g of hot water.

The coffee composition given to the subjects is listed in Table 3.

TABLE 3

| Materials | | Com. | Com. | Com. | Ex. | Ex. |
|---|---|---|---|---|---|---|
| (g) | Manufacturer | Ex. 1 | Ex. 2 | Ex. 3 | 1 | 2 |
| Coffee | Dongsuh Food | 1.6 | 1.6 | 1.6 | 1.6 | 1.6 |
| Sugar | CJ Cheiljedang | 5 | — | — | — | — |
| Erythritol | Zivogreen | — | 8 | — | — | — |
| D-psicose | CJ Cheiljedang | — | — | 9 | 8 | 4 |
| Digestion-resistant | Matsudani Korea | — | — | — | 5.6 | 3.5 |
| maltodextrin | Ltd. | | | | | |
| Rebaudioside A | GLG | — | — | — | — | 0.015 |

(2) Measurement of Changes in Blood Glucose Levels

In order to measure changes in blood glucose after the meal, blood glucose level before meal was checked, followed by providing the subjects with the meal and then coffee was given. Blood glucose was measured for 2 hours at intervals of 30 minutes.

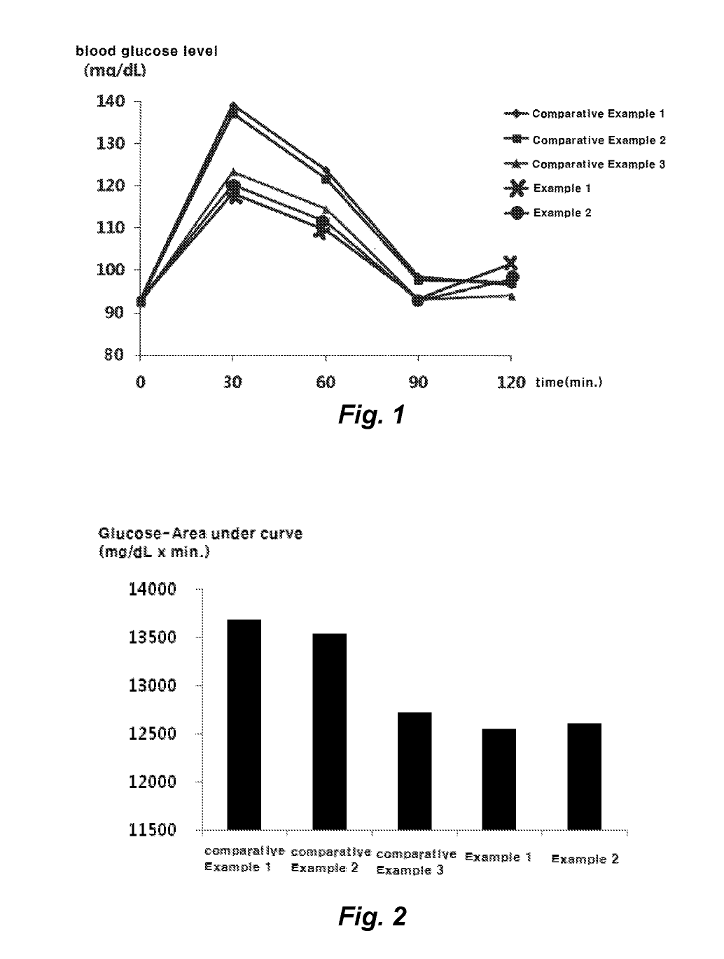

The changes in blood glucose level after meal and coffee intake are summarized in Table 4 (see FIG. 1).

TABLE 4

| | Time | 0 (before | | | | |
|---|---|---|---|---|---|---|
| | (min) | meal) | 30 | 60 | 90 | 120 |
| Blood | Com. Ex. 1 | 93.1 | 139.1 | 123.7 | 98.5 | 96.7 |
| glucose | Com. Ex. 2 | 92.8 | 137.1 | 121.6 | 97.7 | 97.2 |
| (mg/dL) | Com. Ex. 3 | 92.4 | 123.3 | 114.5 | 93.1 | 94.1 |
| | Ex. 1 | 92.9 | *118.3 | *109.5 | *93.4 | 101.6 |
| | Ex. 2 | 93.2 | *120.3 | *111.5 | *92.9 | 97.9 |

*corresponding to time zones in Examples 1 and 2 indicating significant difference of p < 0.01 or less in blood glucose as compared with Comparative Example 1 (sugar).

### Experimental Example 2

### Measurement of Changes in Area Under the Curve of Blood Glucose

The same experiment as in Experimental Example 1 was performed for 2 hours at an interval of 30 minutes to measure G-AUC (Glucose-Area under curve) of the subjects.

The results of G-AUC are summarized in Table 5.

TABLE 5

| | | Time (min) | | | |
|---|---|---|---|---|---|
| | | 30 | 60 | 90 | 120 |
| G-AUC | Comparative | 3483 | 7425 | 10758 | 13686 |
| (mg/dL × min) | Example 1 | | | | |
| | Comparative | 3449 | 7329 | 10619 | 13542 |
| | Example 2 | | | | |
| | Comparative | 3236 | 6803 | 9917 | 12725 |
| | Example 3 | | | | |
| | Example 1 | 3168 | 6585 | 9629 | 12554 |
| | Example 2 | 3203 | 6680 | 9746 | 12608 |

As shown in Table 5, both Examples 1 and 2 showed significant decrease in G-AUC for 120 minutes as compared with the composition of Comparative Example 1 (see FIG. 2).

As can be seen from the results of Experimental Examples 1 and 2, increase in post-meal blood glucose persisted for about 60 minutes and 60 minutes post-meal, the blood glucose level showed a tendency to return to fasting blood glucose level.

When the subjects drank coffee containing the sweetener composition after the meal, increase in blood glucose to 60 minutes after drinking coffee was high in order of Comparative Example 1, Comparative Example 2, Comparative Example 3, Example 2, and Example 1. At 90 minutes to 120 minutes after having the meal and drinking the coffee, the blood glucose levels in Examples 1 and 2 were higher than Comparative Examples 1 to 3.

One of the main causes for diabetes is poor ability to control insulin, which leads to some problems. That is, sharp rise in the blood glucose level after the meal occur, causing excessive secretion of insulin, and the fasting blood glucose level causes hypoglycemic conditions.

Comparative Example 1 showed the highest increase in blood glucose resulting from the combined increase in blood glucose due to the meal and the coffee containing sugar. Comparative Example 2 showed increase in blood glucose due to the meal only. Comparative Example 3 showed that D-psicose has an effect of lowering increase in blood glucose after the meal.

It could be seen that the compositions of Examples 1 and 2 inhibited sharp rise in post-meal blood glucose and showed increase in fasting blood glucose as compared with the composition of Comparative Examples. It can be understood that the composition of Example 1 provided an effect of preventing hypoglycemia on an empty stomach or after a certain period of time has elapsed from the meal by consistently supplying blood glucose while inhibiting sharp change in blood glucose.

In other words, in Examples 1 and 2, it is determined that the carbohydrate absorption inhibition effect in the small intestine owing to D-psicose and the food digestion delay effect owing to the digestion-resistant maltodextrin may significantly lower a sharp rise in post-meal blood glucose, which allows slow digestion of food, thereby consistently supply the body with saccharides even 2 hours after the meal.

### Experimental Example 3

### Sensory Evaluation of Sweetener Composition

In order to evaluate sensory perception of the sweetener compositions prepared in Examples 1 and 2, sensory evaluation was performed on 25 adult males and females in their twenties to fifties using coffee each containing sucrose prepared in the same manner as in Experimental Example 1 (corresponding to Comparative Example 1 of Experimental Example 1) and coffee each containing the sweetener composition of Examples 1 and 2.

Sensory analysis was evaluated on a scale of 1 to 5. Results of the sensory analysis are summarized in Table 6.

TABLE 6

| | General | Color | General | | |
|---|---|---|---|---|---|
| | accept- | accept- | flavor | Taste | Aftertaste |
| | ability | ability | acceptability | acceptability | acceptability |
| Coffee | 3.30 | 3.62 | 3.52 | 3.34 | 3.22 |
| containing | | | | | |
| sugar | | | | | |
| Example 1 | 3.15 | 3.50 | 3.35 | 3.30 | 3.20 |
| Example 2 | 3.28 | 3.55 | 3.47 | 3.20 | 3.24 |

As can be seen from the results of Experimental Examples 1 to 3, it is determined that the sweetener composition of the present invention has sweetness and sensory quality similar to sugar and an effect of preventing sharp rise in blood glucose and fasting hypoglycemic condition. Therefore, the sweetener composition corresponds to an effective sweetener for diabetic patients. Furthermore, the sweetener composition may be applied in various ways for persons requiring control/management of blood glucose.

1. A method for preventing hyperglycemic and hypoglycemic symptoms, comprising administering a sweet composition containing D-psicose and a slowly digestible polysaccharide or a digestion-resistant polysaccharide as active ingredients, to the subject.

2. The method as claimed in claim 1, wherein the sweetener composition prevents hyperglycemic symptom after meal and hypoglycemic symptom on an empty stomach.

3. The method as claimed in claim 1, wherein the slowly digestible polysaccharide or the digestion-resistant polysaccharide comprises at least one polysaccharide selected from the group consisting of palatinose, trehalose, digestion-resistant maltodextrins, and oligosaccharides.

4. The method as claimed in claim 1, wherein the sweet composition further comprises a high-intensity sweetener.

5. The method as claimed in claim 4, wherein the sweet composition comprises at least one high-intensity sweetener selected from the group consisting of steviol glycoside, sucralose, aspartame, Siraitia grosvenori extract, Glycyrrhiza uralensis Fischer extract, thaumatin, and agave syrup.

6. The method as claimed in claim 4, wherein the slowly digestible polysaccharide or the digestion-resistant polysaccharide is present in an amount of 0.01 to 200 times based on the weight of D-psicose, and the high-intensity sweetener is present in an amount of 0.001 to 2 times based on the weight of D-psicose.

7. The method as claimed in claim 2, wherein a level of blood glucose after meal in the subject for preventing the hyperglycemic and the hypoglycemic symptoms ranges from fasting blood glucose level to less than 130% of the fasting blood glucose level.

8. The method as claimed in claim 1, wherein the sweet composition is administered after a meal.

9. The method as claimed in claim 2, wherein the after meal means within 1 hour after a meal.

10. The method as claimed in claim 2, wherein the empty stomach means 2 hours after a meal.

11. The method as claimed in claim 1, wherein the sweet composition is provided as a beverage.
